(19) United States
(12) Patent Application Publication
(10) Pub. No.: US 2005/0228281 A1
(43) Pub. Date: Oct. 13, 2005

(54) Handheld diagnostic ultrasound system with head mounted display

(76) Inventor: Thomas P. Nefos, Fort Collins, CO (US)

Correspondence Address: Thomas P. Nefos, 2300 West County Road 38E, #175, Fort Collins, CO, 80526, US

(21) Appl. No.: 10/815,077

(22) Filed: Mar. 31, 2004

(51) Int. Cl. A61B 8/00

(52) U.S. Cl. 600/446

(57) ABSTRACT

A handheld diagnostic ultrasound device is disclosed which is pocket sized, lightweight and is designed for clear imaging under adverse lighting conditions by utilizing a custom Head Mounted Display (HMD). The handheld diagnostic ultrasound device comprises a removable probe and a removable HMD that connects to the personal digital assistant (PDA) style control unit. The handheld diagnostic ultrasound device may be viewed on the VGA LCD Display on the diagnostic ultrasound device or on the HMD. Images obtained may be stored to a secure digital flash or transferred via USB (Universal Serial Bus) or wireless link to a remote computer system for further processing or archiving. The handheld diagnostic ultrasound device is based on a dual-core processor that achieves a level of integration not previously attainable in a handheld diagnostic ultrasound device.

### RELATED APPLICATIONS

The present application is related to U.S. Pat. No. 6,641,533, issued Nov. 4, 2003, for HANDHELD PERSONAL DATA ASSISTANT (PDA) WITH A MEDICAL DEVICE AND METHOD OF USING THE SAME, by Causey, III et al., included by reference herein.

The present application is related to U.S. Pat. No. 6,569,102, issued May 27, 2003, for MINIATURIZED ULTRASOUND APARATUS AND METHOD, by Imran et al., included by reference herein.

The present application is related to U.S. Pat. No. 6,569,101, issued May 27, 2003, for MEDICAL DIAGNOSTIC ULTRASOUND INSTRUMENT WITH ECG MODULE, AUTHORIZATION MECHANISM AND METHODS OF USE, by Quistgaard et al., included by reference herein.

The present application is related to U.S. Pat. No. 6,540,685, issued Apr. 1, 2003, for ULTRASONIC DIAGNOSTIC DEVICE, by Rhoads et al., included by reference herein.

The present application is related to U.S. Pat. No. 6,540,682, issued Apr. 1, 2003, for PORTABLE, CONFIGURABLE AND SCALABLE ULTRASOUND IMAGING SYSTEM, by Leavitt et al., included by reference herein.

The present application is related to U.S. Pat. No. 6,530,887, issued Mar. 11, 2003, for ULTRASOUND PROBE WITH INTEGRATED ELECTRONICS, by Gilbert et al., included by reference herein.

The present application is related to U.S. Pat. No. 6,514,205, issued Feb. 4, 2003, for MEDICAL DIGITAL ULTRASONIC IMAGING APPARATUS CAPABLE OF STORING AND REUSING RADIO-FREQUENCY(RF) ULTRASOUND PULSE ECHOES, by Lee et al., included by reference herein.

The present application is related to U.S. Pat. No. 6,497,644, issued Dec. 24, 2002, for MEDICAL DIAGNOSTIC ULTRASOUND SYSTEM AND METHOD, by Randall et al., included by reference herein.

The present application is related to U.S. Pat. No. 6,379,304, issued Apr. 30, 2002, for ULTRASOUND SCAN CONVERSION WITH SPATIAL DITHERING, by Gilbert et al., included by reference herein.

The present application is related to U.S. Pat. No. 6,106,472, issued Aug. 22, 2000, for PORTABLE ULTRASOUND IMAGING SYSTEM, by Chiang et al., included by reference herein.

The present application is related to U.S. Pat. No. 5,690,114, issued Nov. 25, 1997, for PORTABLE ULTRASOUND DEVICE, by Chiang et al., included by reference herein.

The present application is related to U.S. Pat. No. 5,197,037, issued Mar. 23, 1993, for METHOD AND APPARATUS FOR THE SIMULTANEOUS PERFORMANCE OF THE BEAM FORMATION AND SCAN CONVERSION IN A PHASED ARRAY SYSTEM, by Leavitt, included by reference herein.

Jensen, Jorgen Arendt, “Implementation of ultrasound time-domain cross-correlation blood velocity estimators”

IEEE Transactions on Biomedical Engineering, Vol. 40, no 5, pp 468-474, 1993

Chihiro Kasai, Koroku Namekawa, Akira Koyano and Ryozo Omoto, “Real-Time Two-Dimensional Blood Flow Imaging Using an Autocorrelation Technique”, IEEE Transactions on Sonics and Ultrasonics, VOL SU-32, NO 3, May 1985

B. G. Tomov and J. A. Jensen, “A new architecture for a single-chip multi-channel beamformer based on a standard FPGA”,

Proc IEEE Ultrason. Symp, pp 1529-1532, 2001

Siddhartha Sikdar, Ravi Managuli, Lixin Gong, Vijay Shamdasani, Tsuyoshi Mitaje, Tetsuya Hayashi and Yongmin Kim, “A Single Media processor-Based Programmable Ultrasound System”,

IEEE Transactions on Information Technology in Biomedicine, VOL 7, NO 1, March 2003

Siddhartha Sikdar, Ravi Managuli, and Yongmin Kim, “Scan Conversion for a Multi-processor-based Ultrasound Processing System”,

Proceedings of SPIE VOL 4681 (2002)

Sverre Holm and Hongxia Yao. Improved frame rate with synthetic transmit aperature imaging using prefocused subaperatures,

IEEE Ultrasonics Symposium, October 1997

### FIELD OF THE INVENTION

The present invention relates to ultrasonic devices and specifically to a handheld diagnostic ultrasound device, which is lightweight, portable and is designed for use in adverse lighting conditions by use of a custom Head Mounted Display (HMD)

### BACKGROUND OF THE INVENTION

Portable ultrasound devices are known in the medical and veterinary communities but many of these devices suffer from the problem of viewing the image under adverse lighting conditions as may exist outdoors, such as, full sunlight. Standard CRT or LCD Display devices are difficult to operate at night, when the controls to operate the device cannot be seen. The Head Mounted Display (HMD) overcomes these problems by providing a consistent viewing environment and by providing the sonographer visual feedback of all system functions.

EMS professionals are in need of such a diagnostic ultrasound device that allows true field use, as there may not be a suitable surface on which to place a typical LCD Display or CRT type unit. The EMS attendant needs hands free operation to attend to the victim. In battlefield conditions, freedom of movement and split-second diagnosis is of paramount importance. Since a standard display light source could become a target for the battlefield practitioner, a diagnostic ultrasound device should allow the operator to select HMD only to conserve power and minimize light detection. In small clinics or triage environments the ability to recall patient images from a database, as well as image transfer options like wireless link, USB (Universal Serial Bus) or removable secure digital flash storage, permit ease of image manipulation. The JPEG (Joint Photographic Encryption Group) standard has now replaced expensive DICOM (Digital Imaging and Communications in Medicine) standard for many users. In all cases, the ability to have a hands free diagnostic ultrasound device is of paramount importance.

A prior art example in U.S. Pat. No. 6,540,685 (Rhoads et al.), which shows a figure of a doctor with a diagnostic ultrasound device strapped around her neck. This would not allow hands free use as the diagnostic ultrasound device would swing out of view as the practitioner bends down to scan a victim on the ground. The unit would also not be “sunlight readable” and even in good lighting conditions, the viewing angle would be limited.

Another prior art example U.S. Pat. No. 6,569,102 Imran et al.), which shows a miniaturized ultrasound device that would not be useable under adverse lighting conditions such as direct sunlight. Snap-on probe heads further limit the viewing angle of the display, as the probe head requires the viewing display to be at specific scanning angle, not optimum viewing angle.

Another prior art example U.S. Pat. No. 6,530,887 (Gilbert et al.), which describes a portable ultrasound device based on an IEEE 1394 “fire wire” interface to a laptop computer. This unit also suffers from the fact that it is relatively large and heavy as it is designed around a laptop computer system. To see the image with this or any “clam-shell” device, the device must be opened. This takes additional space and provides limited freedom of movement by the sonographer.

These are examples of deficiencies of the known diagnostic ultrasound devices, as they exist today. None of these devices provides the constancy of visual display viewing angle or ease of operation in all lighting conditions that a Head Mounted Display (HMD) would offer the sonographer. A need exists for a portable, handheld diagnostic ultrasound device that is pocket sized and overcomes limitations of known diagnostic ultrasound devices for field use in adverse lighting conditions.

It is therefore an object of the invention to provide a lightweight, portable, handheld, low power, diagnostic ultrasound device.

It is another object of the invention to allow use of such a device in adverse lighting conditions via a custom Head Mounted Display (HMD).

It is another object of the invention to provide an HMD that allows constancy of viewing angle, speakers for audio reception of Doppler mode, and an electret condenser microphone that records the sonographer's speech as an audio tag associated with an image.

It is another object of the invention to provide an HMD where the speakers are “ear speaker” style in that they are inserted in the ear and reduce ambient noise.

It is another object of the invention to provide an HMD that has an electret condenser microphone that allows clear speech from the patient to be heard in the ear speaker so that conversation is unimpeded in a natural manner.

It is another object of the invention to provide an HMD that allows a virtual 4-foot view of the ultrasound image in both eyes so as not to fatigue the eyesight of the sonographer.

It is another object of the invention to provide an HMD with improved detail of anatomical structure that can be seen under all lighting conditions as the HMD provides a constant viewing
angle and environment.

It is another object of the invention to provide a device with which a doctor can review the image of the last visit to the clinic, to determine patient progress.

It is another object of the invention to provide a keypad that provides one button access to diagnostic ultrasound device functions.

It is another object of the invention to provide a device where all operating information is confirmed displayed and fed back to the operator through the HMD such that the operator does not have to look at the control unit for visual feedback.

It is another object of the invention to provide a probe that contains a freeze button so that any active image can be held on the display for closer inspection or measurement.

It is another object of the invention to provide a dual-core processor that controls all diagnostic ultrasound device functions and is a highly integrated, low power “system-on-a-chip” (SOC).

It is another object of the invention to provide a dual-core processor that allows functional upgrades and new features for the diagnostic ultrasound device in the field.

It is another object of the invention to provide removable probes for various diagnostic requirements including, but not limited to: phased array, linear, curve linear and mechanical sector.

### SUMMARY OF THE INVENTION

In accordance with the present invention, there is provided a handheld diagnostic ultrasound device for acquiring and displaying ultrasonic images. The device includes a Head Mounted Display (HMD) that allows constancy of viewing angle, speakers for audio reception of Doppler mode, and an electret condenser microphone that records the sonographer's speech as an audio tag associated with an image. The speakers are “ear speaker” style, inserted in the ear, reducing ambient noise. The electret condenser microphone allows clear speech from the patient to be heard in the ear speaker so that conversation is unimpeded. The HMD allows a virtual 52″ view of the ultrasound image in both eyes so as not to fatigue the eyesight of the sonographer. Improved detail of anatomical structure that can be seen under all lighting conditions as the HMD provides a constant viewing angle and environment. A doctor can review the image of the last visit to the clinic to evaluate patient progress. For instance, the handheld diagnostic ultrasonic device could be worn on a belt or in a pocket such that the controls and cable exit would be in the upright position, with the handheld VGA display turned off to conserve power. The sonographer would have the HMD device around his neck in the manner of a stethoscope until he examines the patient. The sonographer would then wear the HMD. The lower one third of the device is of a clear protective nature so that vision is minimally impaired and the sonographer is free to walk normally. The probe is attached via a small clip to the pocket or other clothing until needed. When the sonographer unclips the probe for use, only the probe occupies the scanning hand. The other hand is free to interact with the patient.

The device has four rows of buttons (keypad) plus a trackball that can be operated as needed with one hand. The controls are similar to a cellular phone for numerical and text entry. However, they also provide one button access to diagnostic ultrasound device functions. The buttons may be optionally backlit with a light sensor controlling the backlight as needed. The trackball can be operated by finger touch with position of the cursor displayed on the HMD.

All device operating information is confirmed displayed and fed back to the operator through the HMD such that the operator does not have to look at the control unit for visual feedback. The probe contains a freeze button so that any active image can be held on the display for closer inspection or measurement. The dual-core processor controls all diagnostic ultrasound device functions and is a highly integrated, low power “system-on-a-chip” (SOC). The dual-core processor allows functional upgrades and new features for the diagnostic ultrasound device in the field.

The diagnostic ultrasound device supports removable probes for various diagnostic requirements including, but not limited to: phased array, linear, curve linear and mechanical sector. Mechanical sector probes allow an inexpensive single crystal, motor swept field of view for a very affordable ultrasonic system. Mechanical probes, however, are not appropriate for Doppler modes as will be known to those familiar with the art. The phased array probe produces a similar sector image with predictability of transmit/receive angle required for continuous wave Doppler (CWD), pulse wave Doppler (PWD), power Doppler (PD) or Color Flow Doppler (CFD). The phased array probe allows beam steering as may be required for chest cavity or cardio diagnostic use. Another probe, the linear, is very slender in shape to allow rectal use. A broad range of probes is accommodated to allow flexible use of the ultrasound diagnostic device.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective top view of a diagnostic_ultrasound_device in accordance with the present invention;

FIG. 2 is a perspective view of a key_pad in accordance with the present invention;

FIG. 3 is a perspective bottom view of a diagnostic_ultrasound_device in accordance with the present invention;

FIG. 4 is a complete system view of a diagnostic_ultrasound_device in accordance with the present invention;

FIG. 5 is an application view of a diagnostic_ultrasound_device in accordance with the present invention;

FIG. 6 is an application view of a diagnostic_ultrasound_device in accordance with the present invention;

FIG. 7 is a perspective view of a head_mounted_display (HMD) of the diagnostic_ultrasound_device in accordance with the present invention;

FIG. 8 is a block diagram view of a diagnostic_ultrasound_device in accordance with the present invention;

FIG. 9 is a block diagram view of a dual-core_processor in accordance with the present invention;

FIG. 10 is a block diagram view of a pixel point beamformer in accordance with the present invention;

FIG. 11 is a block diagram view of a power_supply and battery charger in accordance with the present invention; and

FIG. 12 is a block diagram view of a doppler subsystem in accordance with the present invention.

For purposes of clarity and brevity, like elements and components will bear the same designations and numbering throughout the FIGURES.

### DESCRIPTION OF THE PREFERRED EMBODIMENT

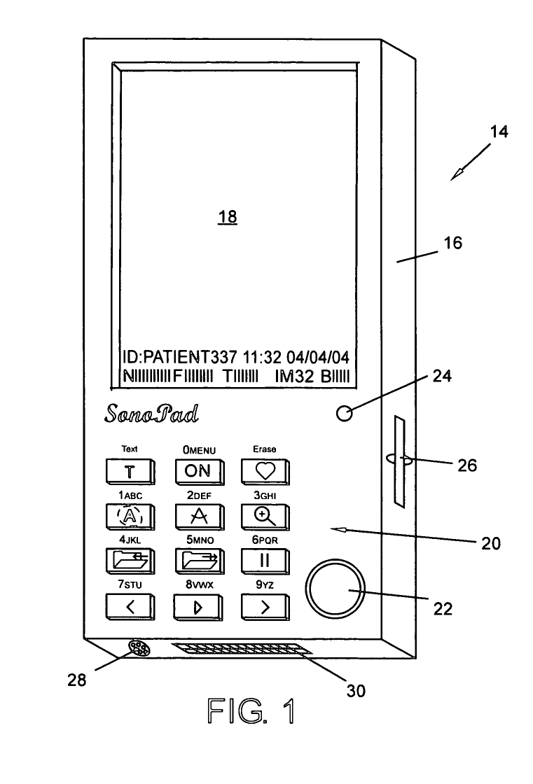

FIG. 1 is a perspective top view of a diagnostic_ultrasound_device in accordance with the present invention.

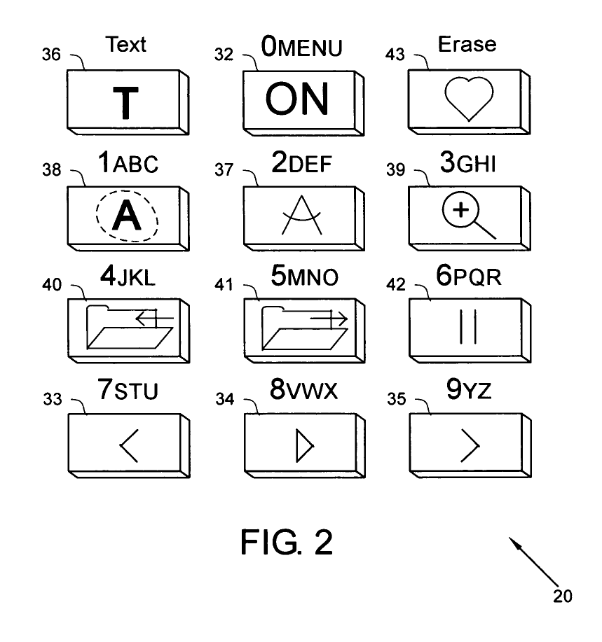

FIG. 2 is a perspective view of a key_pad in accordance with the present invention.

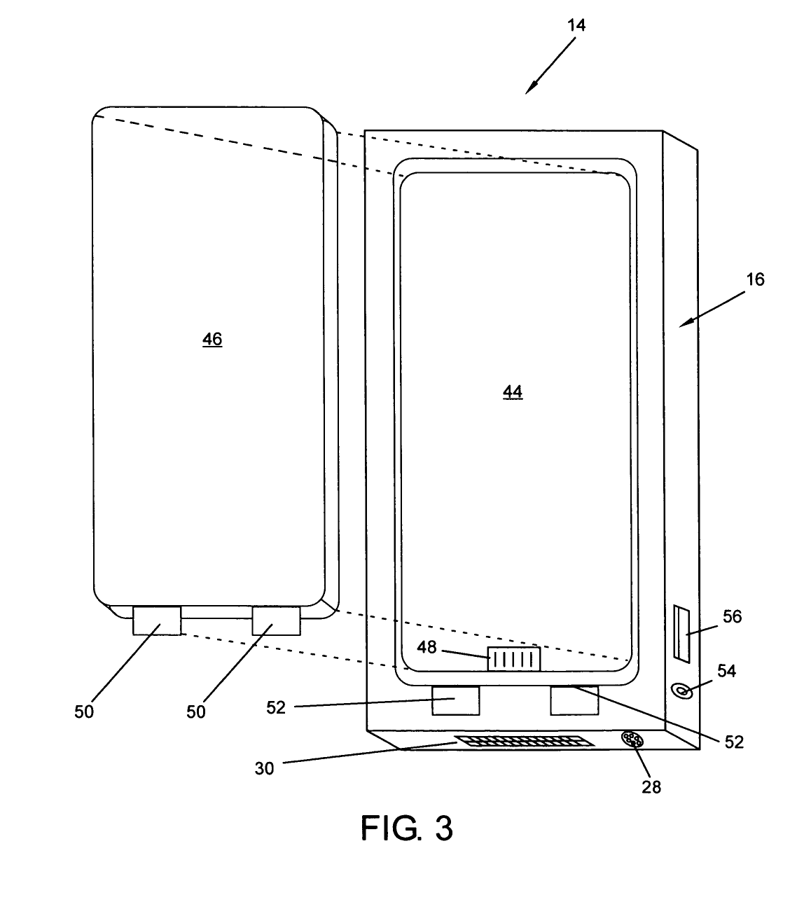

FIG. 3 is a perspective bottom view of a diagnostic_ultrasound_device in accordance with the present invention.

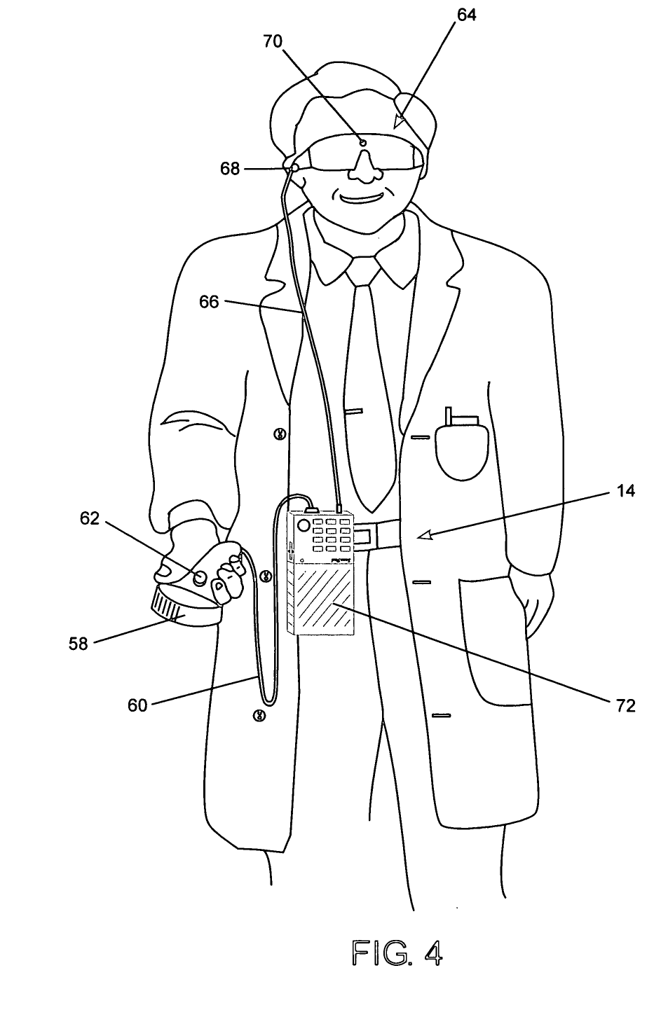

FIG. 4 is a complete system view of a diagnostic_ultrasound_device in accordance with the present invention.

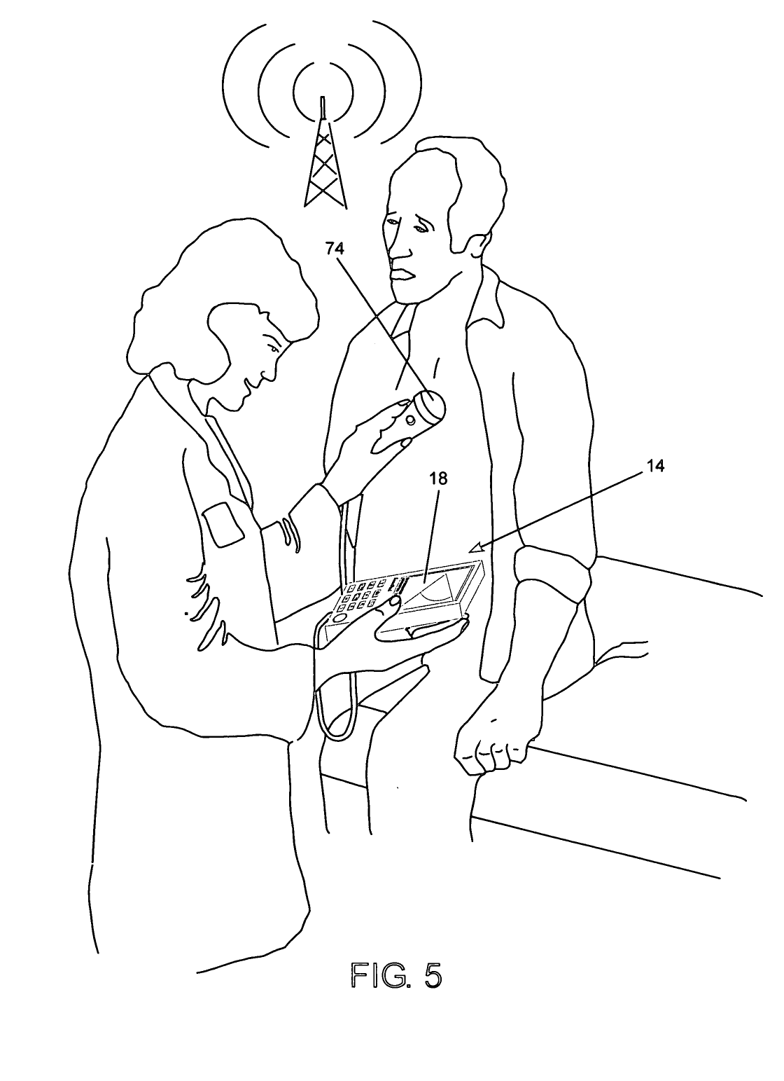

FIG. 5 is an application view of a diagnostic_ultrasound_device in accordance with the present invention.

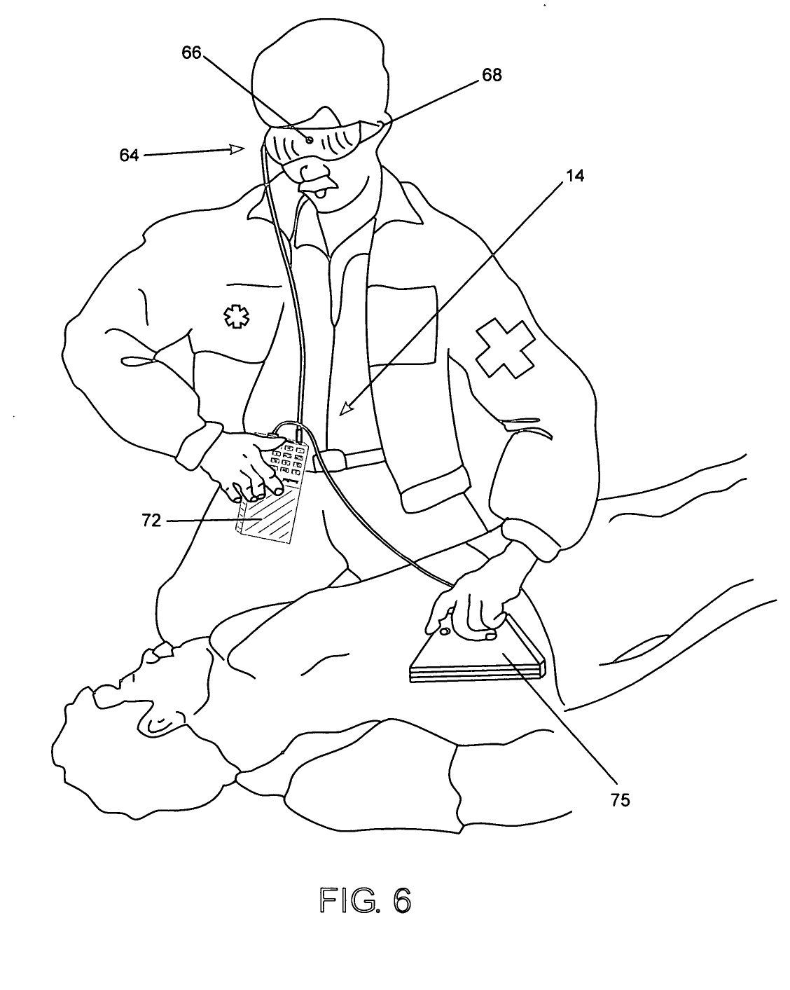

FIG. 6 is an application view of a diagnostic_ultrasound_device in accordance with the present invention.

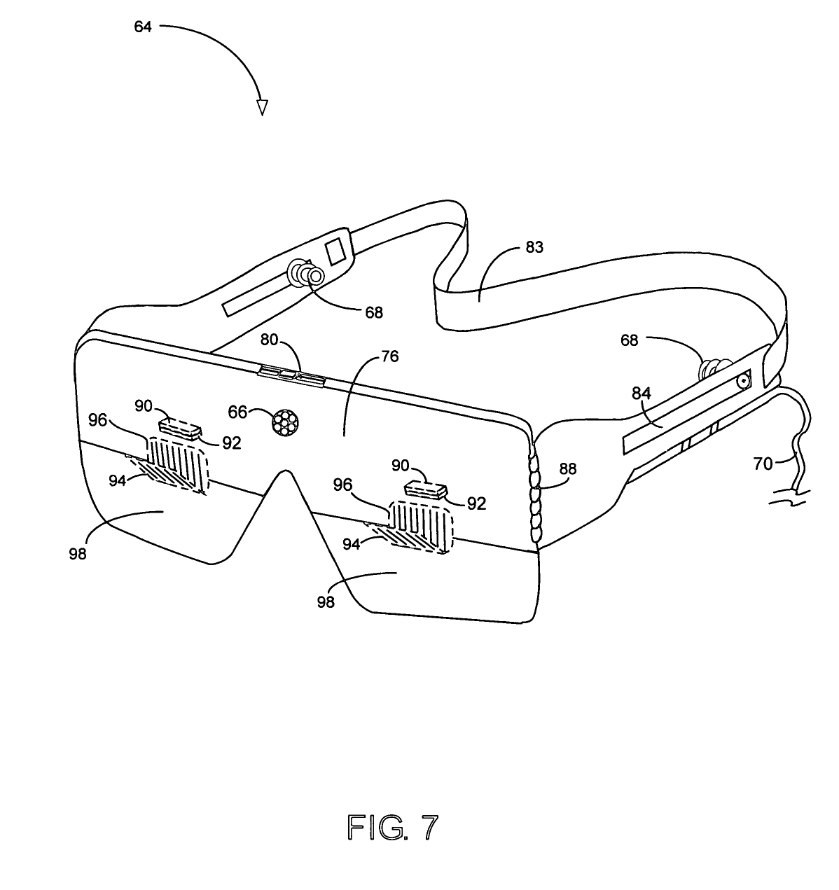

FIG. 7 is a perspective view of a head_mounted_display (HMD) of the diagnostic_ultrasound_device in accordance with the present invention.

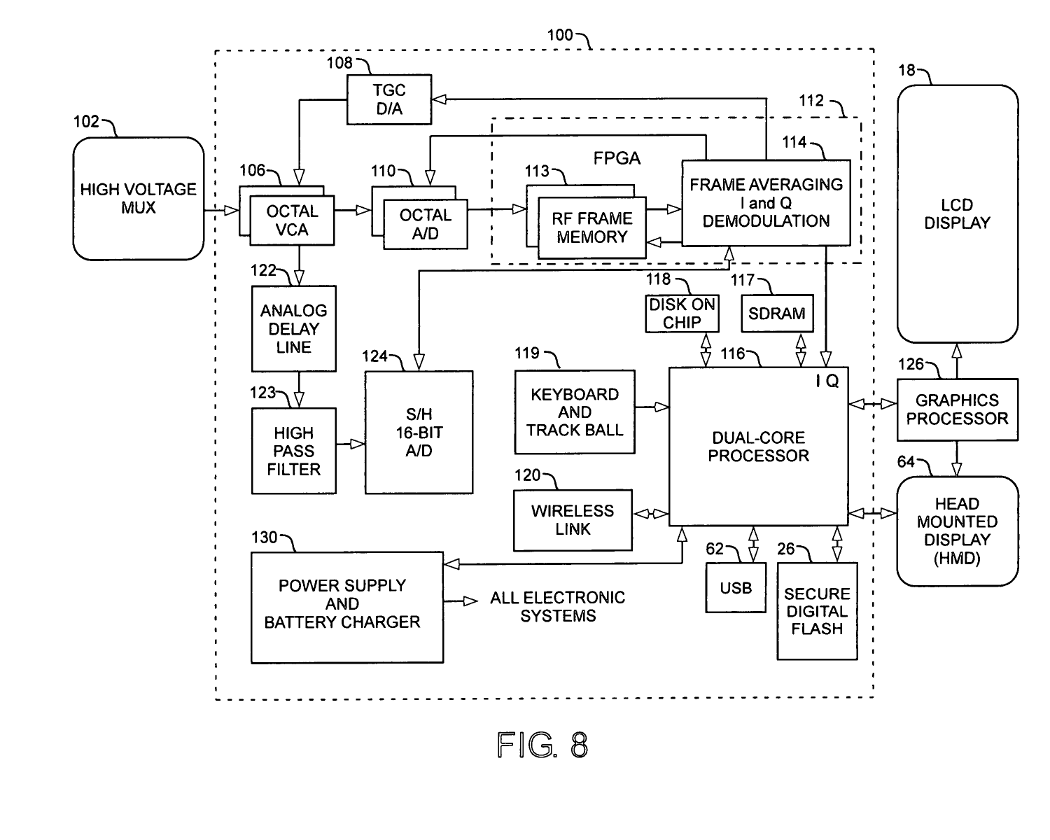

FIG. 8 is a block diagram view of a diagnostic_ultrasound_device in accordance with the present invention.

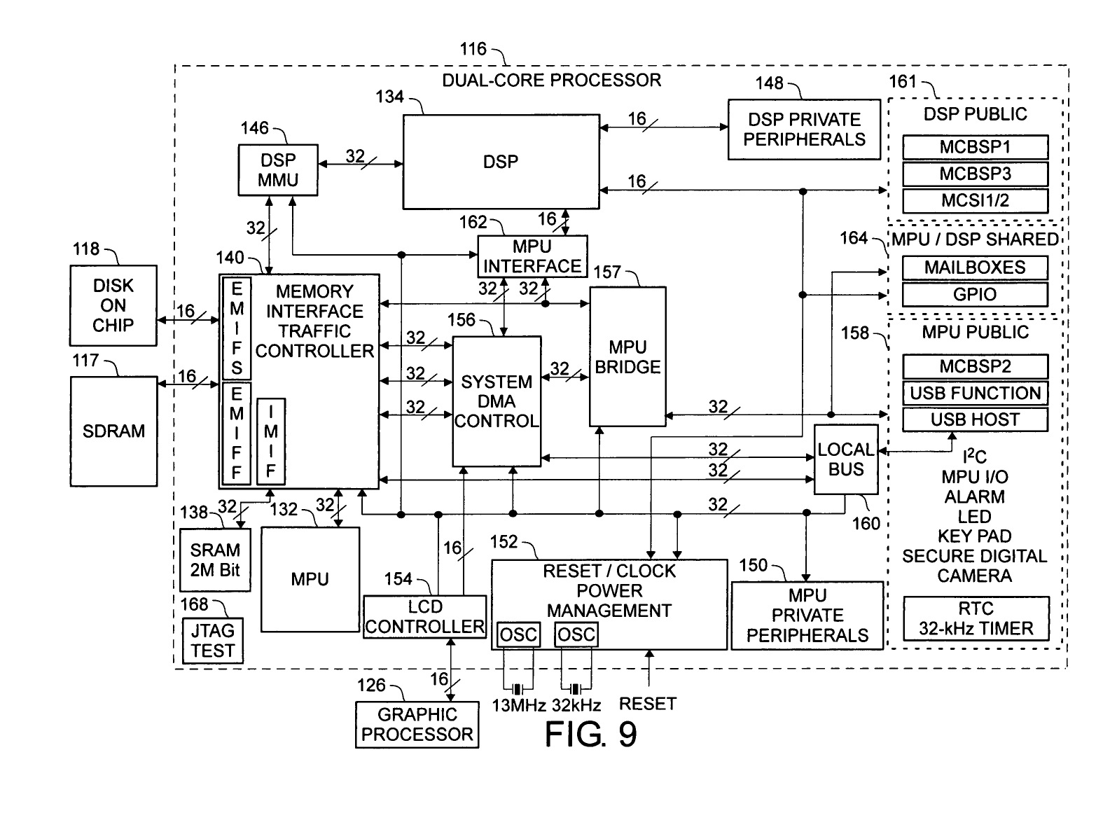

FIG. 9 is a block diagram view of a dual-core_processor in accordance with the present invention.

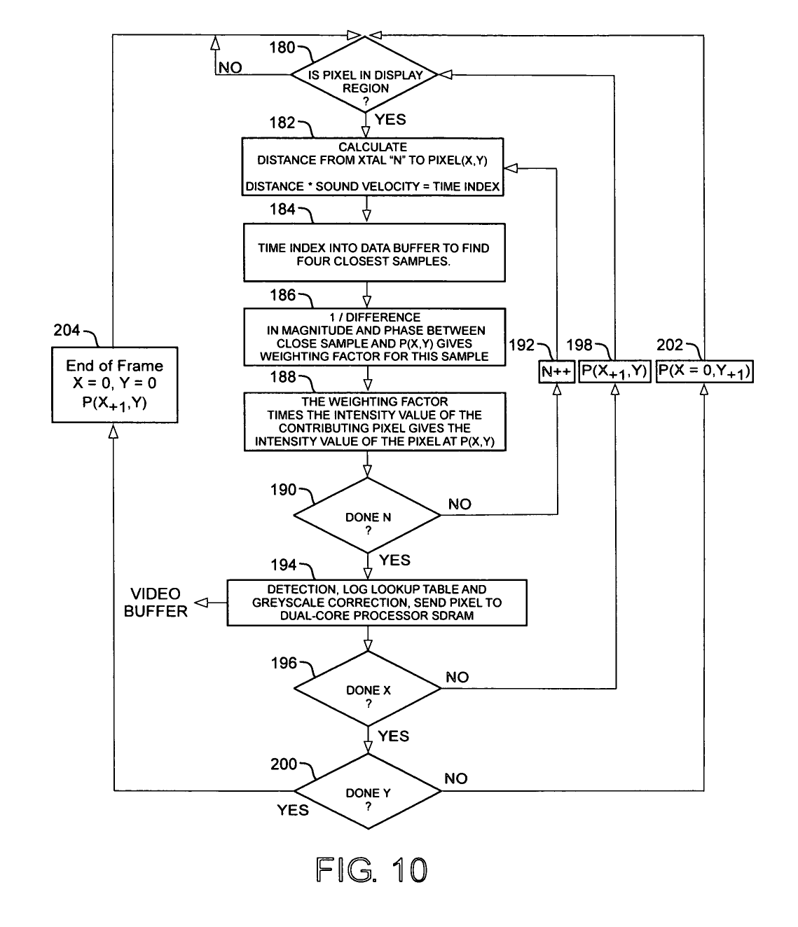

FIG. 10 is a block diagram view of a pixel point beamformer in accordance with the present invention.

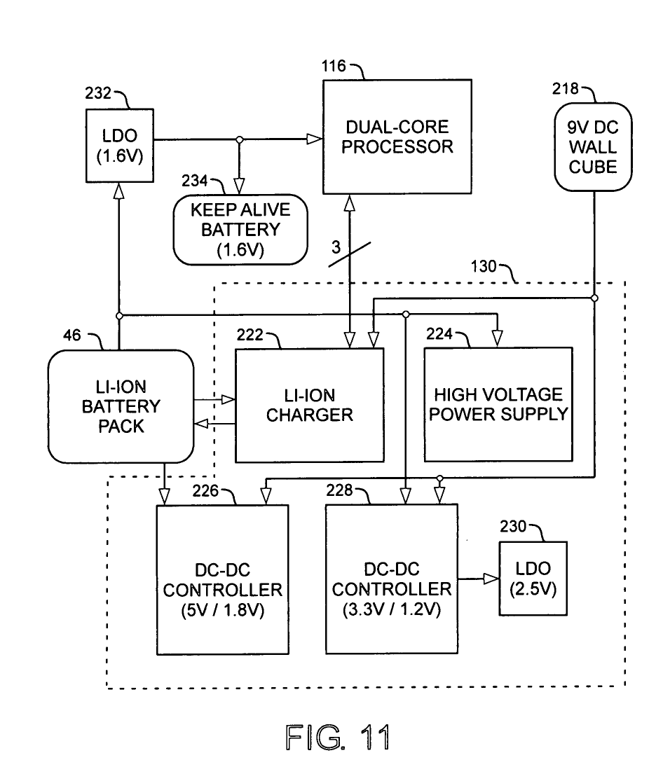

FIG. 11 is a block diagram view of a power_supply and battery charger in accordance with the present invention.

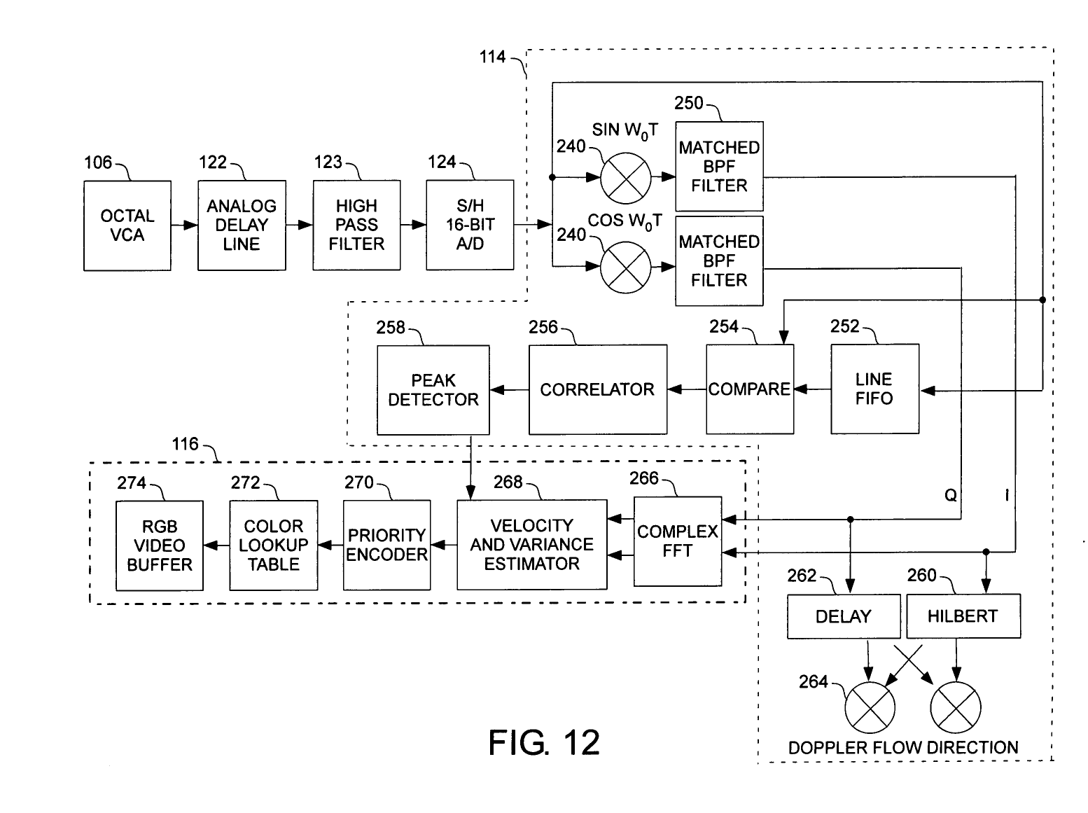

FIG. 12 is a block diagram view of a doppler subsystem in accordance with the present invention.

### DETAILED DESCRIPTION OF THE INVENTION

FIG. 1 is a topside pictorial representation of the diagnostic ultrasound device 14 in accordance with the preferred embodiment of the present invention. The diagnostic ultrasound device 14 is preferably seven inches long, three inches wide and one inch thick. Those skilled in the art will understand that the present disclosure is not limited to these exact dimensions or form. The weight of the diagnostic ultrasound device 14 is preferably less than two pounds. The system case 16 is made of reinforced molded nylon and rubber material for durability. The material can withstand a one-meter drop to a concrete surface without damage to the diagnostic ultrasound device 14.

The VGA display on the diagnostic ultrasound device 14 is a LCD display 18 with 640×480×16-bit per pixel resolution. Such a LCD display 18 is typified by the low power Toshiba VGA display, Model LTM04C380K. Display technology is rapidly improving, so more power efficient models or higher resolution may be accommodated as will be understood by those familiar with the art. The LCD display 18 shows:
1) Patient ID
2) Time in Military Format
3) Date in Format set by Setup Menu
4) Near Gain Numerical Display
5) Far Gain Numerical Display
6) TGC Gain Numerical Display
7) Current Image Number Recalled
8) Battery Charge Numerical Display
9) Current Mode of Operation

The VGA display is protected by a clear protective surface, polarized to minimize sunlight reflection and optically coated to enhance brightness and contrast. The optical enhancement process allows maximum lumen output while keeping the CCFL or LED 24 backlight to a minimum setting to preserve power.

A keypad 20 is provided for all user entry and control of the diagnostic ultrasound device 14. The keypad 20 is constructed such that it may be backlit for handheld use in adverse lighting conditions. The keypad 20 is designed to be cleaned with common anti-bacterial and anti-fungal cleaning agents such as Novasan.

A trackball 22 device is provided for caliper measurements, area measurements zoom box positioning and Doppler area of interest. This trackball 22 device has a scraper ring (not shown) that keeps the ball (not shown) free of contaminants. The ring and ball may be removed from the front of the diagnostic ultrasound device 14 for cleaning. The trackball 22 device allows an operator to position a cursor without looking at the diagnostic ultrasound device 14 itself when using the head_mounted display.

The diagnostic ultrasound device 14 provides a led 24 and an audible alarm (not shown). The audible alarm provides tactile feedback of the keypad 20 and system low battery indication. The led 24 can be blue, green or orange. When the ultrasound device is “ON”, the led 24 is illuminated blue. If the battery is low, the led 24 is illuminated orange. When the battery pack is charging, the led 24 pulses orange until the battery pack (not shown) is charged, then the led 24 is illuminated green, indicating a full charge. The diagnostic ultrasound device 14 must be “OFF” to allow the battery pack to be charged. The HMD, herein below described, displays the battery status, which shows the operator how much electric charge, remains. When the battery pack is depleted to 10% of its nominal capacity, the audible alarm, if enabled in the setup menu, periodically beeps, to let the user know the battery is low. This beep is also heard in the ear-speaker, herein below described.

A removable secure digital flash card 26 is the only non-volatile image storage on the handheld diagnostic ultrasound device 14. When an image is recalled for display, it is de-compressed and displayed on the HMD and/or LCD Display 18. Each single image has a five second audio tag stored on the card as well. If the audio tagging is turned “ON” in the setup menu, the individual images can be stored at a five second rate, whereas a 64 sequence image (Cineloop™) is stored at the frame rate of 30 frames per second. The numbers given for storage are typical, but it should be understood that the secure digital flash card 26 memory is dynamically allocated up the maximum card capacity. The card may be write protected to avoid accidental erasure of the stored images.

An HMD connector 28 (Switchcraft T6M) provides power, ground, composite video, microphone and audio to the HMD via a one-meter six-wire cable. The connection is sensed for HMD present and if selected in the set-up menu, the handheld LCD display 18 is turned off to save power. The composite video is always present via a special six-pin adaptor cable for video output to a video printer, video monitor or videocassette recorder.

The probe connector 30 is a 50 pin, high density, male locking device as typically manufactured by HONDA that allows all probes to be connected to the diagnostic ultrasound device 14. The connection is recessed such that the probe female connector maintains its position perpendicular to the male diagnostic ultrasound device's connector to avoid connector damage. The connector is shielded to minimize noise. The probe connector 30 passes all probe control signals, data signals and operating voltage to the connected probes. The probe connector 30 has a special pin that senses the presence of a probe and will de-energize the probe operating pulses if no probe is sensed. The probe connector 30 has a special “ID” pin that determines what probe is connected to the diagnostic ultrasound device 14. The probe specific settings are stored in flash memory and recalled for use with the specific probe when detected by the “ID” pin.

FIG. 2 describes the keypad 20 in detail, as it exists on the diagnostic ultrasound device 14 of the present invention. The keypad 20 layout on the diagnostic ultrasound device 14 resembles that of a cellular phone where each key represents:
A) A number 0-9;
B) A group of letters;
C) A specific ultrasound function.

The specific keypad 20 functions will now be discussed. The on key 32 allows the diagnostic ultrasound device 14 to turn “ON” and “OFF”. If the on key 32 is pressed quickly, the diagnostic ultrasound device 14 will turn “ON”. If the on key 32 is again pressed quickly while the diagnostic ultrasound device 14 is “ON”, a setup menu will appear on the VGA display. The setup menu may include but is not limited to:
1. Set patient ID
2. Set time and date
3. Set date format
4. Set default probe filter
5. Set RF frame averaging for the probe
6. Set Video frame averaging
7. Set Caliper Conversion Table
8. Set Area Conversion Table
9. Set default measurement table for calipers
10. Set default Doppler mode
11. Set handheld alarm mute
12. Set JPEG or DICOM™ image storage
13. Disable handheld VGA display if HMD detected
14. Disable key_pad auto-illumination
15. Set audio tagging on/off

If the on key 32 is held down over three seconds, the diagnostic ultrasound device 14 will turn “OFF”. When the setup menu is displayed, the bottom row of keys allows previous key 33, select key 34 and next key 35 of a menu item for all menu selections. If a menu item has additional selections, the bottom row of keys will again be used to move to and select the menu item.

The text key 36 allows numbers and text to be placed on the screen. When text key 36 is depressed, the diagnostic ultrasound device 14 freezes the current image and enters text mode. A flashing cursor line will first appear and may be moved by the trackball 22 device to the location of desired text. If an alphanumeric key is pressed quickly, it is the key number; if a alphanumeric key is pressed again, first letter of the series of associated letters is displayed; if a alphanumeric key is pressed again, the second letter of the series of associated letters is displayed; if the alphanumeric key is pressed again, the third letter of the series of associated letters is displayed. The alphanumeric selection will be completed when the text key 36 is depressed. If in text mode the erase key is pressed, the alphanumeric letters and numbers will be erased similar to a backspace key until the erase key is released. If the erase key is held down over three seconds, all text will be erased and the diagnostic ultrasound device 14 will return to normal active imaging mode. At all times, once a mode is to be exited, a three second key press of the associated function key will return the diagnostic ultrasound device 14 to active scanning mode and clear all menus.

For calipers, when the caliper key 37 is depressed the image is frozen. An “X” is displayed and moved to the required measurement position by the trackball 22. When the caliper key 37 is again depressed, the first caliper measurement point is marked and a second “X” is displayed. The trackball 22 device moves the second “X” until at the desired end of measurement position and the caliper key 37 is depressed again. The display is updated to show the distance in millimeters and displays the selected pre-programmed conversion table. If the caliper key 37 is pressed again, a “+ ” appears and follows in similar manner as the first caliper measurement. When the caliper key 37 is held down for three seconds, all calipers are cleared and the active display is restored.

For area, when the area key 38 is depressed the image is frozen. An “X” is displayed and moved to the required perimeter position by the trackball 22. When the area key 38 is depressed again, pixels will be turned full grey scale around the boundary traced by the trackball 22. When the trackball 22 device closes the perimeter of the object boundary, depressing the area key 38 again will display the area of circumference and perimeter distance measured, along with the selected pre-programmed conversion tables. Holding down the area key 38 for three seconds will restore the active image.

For zoom, when the zoom key 39 is pressed the image is frozen. A “zoom” box of 120×120 pixels is displayed. This zoom box is under trackball 22 device control so the user can move the box to the region of interest. When the zoom key 39 is again depressed, the selected area will be displayed full screen and become active again. When the zoom key 39 is depressed for three seconds the full 304×240 pixel ultrasound image will be restored and active.

When the store key 40 is depressed, any active or frozen image will be stored. If the image is in text, area or caliper mode, the measurements will be stored in the image file. If a user goes to store an image or sequence and there is not enough memory, a “Card Full” message will be displayed. If the store key 40 is pressed and the error “Card Full” is displayed, the user will be asked to clear one image or clear all images. If one image is to be cleared, it must be recalled and selected. When it is selected, the erase key will clear that image. If all images are to be cleared, the user must confirm by selecting “YES” or “NO”. In all cases, if the card is write protected, no images will be deleted. A sequence of 64 images or “Cineloop™” will always write over the existing 64 images with a new sequence. One 64-image sequence is the maximum allowed on any card at one time; which is about two seconds of real time ultrasound. All images are stored in QVGA resolution to the secure digital device. Images are acquired and stored in this compact format; however they are expanded or scaled to VGA for display purposes.

When the recall key 41 is depressed, a list of images will be displayed to the screen and the select key 34 may select the image. If the recall key 41 is depressed again, the images will appear in “round-robin” fashion for each press of the recall key 41. When in recall mode, a special Cineloop™ Icon will be shown in the first image position. If this Cineloop™ Icon is selected and the select key 34 depressed, the last 64 images are recalled in a play back loop, similar in function to a VCR recording device. If the Cineloop™ Icon is recalled and the erased, the diagnostic ultrasound device 14 can store a new set of 64 images in rapid selection for playback. These JPEG or DICOM™ images are stored on the secure digital flash device; a typical device would be a secure digital card as offered by Sandisk™ and others.

The freeze key 42 pauses the active image for all modes of use of the diagnostic ultrasound device 14. This key function is also available directly on most probes as single button on the probe body.

When the diagnostic ultrasound device 14 is operating in any active imaging mode, the previous key 33, select key 34 and next key 35 are used to move to and select the NEAR, FAR and TGC gain adjustment. When the required gain is selected, the previous key 33 and next key 35 will decrement or increment the selected gain. This will be indicated as a change of the numerical value on the LCD display 18 as well as the HMD.

When the Doppler key 43 is depressed, the default Doppler mode as selected in the setup menu will be displayed. If a different Doppler mode is desired, the Doppler modes will appear in “round-robin” fashion for each press of the previous key 33 or next key 35 until selected with the select key 34. The active mode will be displayed on the LCD display 18. When the Doppler key 43 is held down for three seconds, Doppler Mode is cleared and the common ultrasound B-Mode display is restored. The Doppler modes will now be discussed.

When CWD (Continuous Wave Doppler) mode is selected, the B-Mode image is frozen and the Doppler box of 120×120 pixels with an “X” in the center will appear. This “X” is moved by the trackball 22 until the region of interest is centered in the box. The select key 34 is pressed to accept the alignment. The two arms of the “X” represent the direction of transmit and receive beams which angle is increased by the previous key 33 for less and the next key 35 for more until the select key 34 is pressed. A visual slider showing the CW Doppler angle (degrees) is displayed on the LCD Display 18. Pressing the Doppler key 43 will start the CWD Doppler mode. A Doppler Gain (db) slider will be present on the LCD display 18. Doppler information for CWD mode will be displayed as a time velocity spectral display or sonogram. Rate of Flow and Pulse will also be displayed. The Doppler audio will be presented at the HMD ear-speaker.

When PWD (Pulse Wave Doppler) mode is selected, the B-Mode image is frozen and the Doppler box of 120×120 pixels with an “X” in the center will appear. This “X” is moved by the trackball 22 until the region of interest is centered in the box. The select key 34 is pressed to accept the region of interest. Visual sliders representing range depth (mm), range length (mm), PRF (Fprf), wall cut-off frequency (Fc) and GAIN (db) will appear. Sliders are adjusted with visual readout of parameters by the previous key 33 for less and the next key 35 for more until the select key 34 is pressed. Pressing the Doppler key 43 will start the PWD Doppler mode. Doppler information for PWD mode will be displayed as a time velocity spectral display or sonogram. Rate of Flow and Pulse will also be displayed. The Doppler audio will be presented at the HMD ear-speaker.

When CPD (Color Power Doppler) mode is selected, the B-Mode image is frozen and the Doppler box of 120×120 pixels with an “X” in the center will appear. This is moved by the trackball 22 until the region of interest is centered in the box. The select key 34 is pressed to accept the region of interest. The Doppler key 43 is again depressed, The B-Mode display is resumed and Color Power Doppler is displayed. A Doppler Gain slider will be present in the LCD Display 18. The Color Power Doppler is computed from the magnitude of the complex Doppler envelope. The Color Power Doppler is displayed over the B-Mode grayscale Image. The shades of yellow to red will be dependent on the amplitude of blood by the integral of the power spectrum as is well known to those familiar with the art. Random noise has a fairly uniform low power, however, and is therefore displayed with a uniform dark color e.g. (dark blue) in the power Doppler image, clearly separated from the high-power Doppler signals from blood flow (displayed in yellow to red).

When CFD (Color Flow Doppler) mode is selected, the Doppler box of 120×120 pixels with an “X” in the center will appear. This is moved by the trackball 22 until the region of interest is centered in the box. The select key 34 is pressed to accept the region of interest. The Doppler key 43 is again depressed and Color Flow Doppler is displayed. A Doppler Gain slider will be present on the LCD Display 18. The Color Flow Doppler is computed from the autocorrelation of the complex Doppler samples. The Color Flow Doppler is displayed over the B-Mode grayscale image. The shades of blue to red will be dependent on estimating the mean frequency, velocity and variance through autocorrelation of blood movement as is well known to those familiar with the art.

FIG. 3 is a bottom side pictorial representation of the diagnostic ultrasound device 14 in accordance with the preferred embodiment of the present invention. The system case 16 has a molded recess or battery well 44 for a removable Li-Ion Battery 46 pack. The Li-Ion Battery 46 pack is of the type that snaps onto a five pin battery connector 48 as typically made by TYCO™ for Smart Battery Pack use and includes charge/discharge limiting mosfets, thermistor temperature monitoring and coulomb counter that are connected via the battery connector 48 to the diagnostic ultrasound device 14. The Li-Ion Battery 46 pack comprises two parallel groups of cells in series providing an 8.4V topology. The cell groups are typically two or more in parallel connection. The circuit on the battery pack device provides a control signal to the mosfets to charge and discharge the Li-Ion Battery 46 pack and transmits charge status information to the host diagnostic ultrasound device 14. The battery pack has two molded nylon battery tabs 50 that allow secure fastening to the two molded nylon battery latches 52 on the diagnostic ultrasound device 14. The Li-Ion Battery 46 pack of the present invention is designed for charging from a 9V DC @1½ amp 2MM DC Input Connector 54. Those skilled in the art will understand that the present invention is not limited to this style of battery pack as technical improvements in capacity, chemistry and form factor become available. A (Universal Serial Bus) USB connection 56 is provided for transferring images to a personal computer, PDA or external storage device.

FIG. 4 shows a typical application of the diagnostic ultrasound device 14 of the present embodiment. The curved array probe 58 is connected to the diagnostic ultrasound device 14 by a 36-wire probe cable 60 specifically chosen for durability and flexibility. The probe freeze button 62 may be depressed on the probe as required for longer viewing of the image. The diagnostic ultrasound device 14 is connected to the head mounted display 64 (HMD) via a 6-wire HMD cable 66 of approximately one meter in length. The head mounted display 64 (HMD) contains audio ear speaker 68 that block out the ambient noise. An electret condenser microphone 70 on the HMD may be used to record the audio tags that are stored and associated with the images as they are recorded. Five seconds of audio per image may be saved. The electret condenser microphone 70 can be mixed with the audio volume by top mounted, slide actuated potentiometer for a balanced blend of voice/ultrasound audio as may be required at the ear speaker 68. A carry pouch 72 is provided for hands free operation of the diagnostic ultrasound device 14. The carry pouch 72 allows the diagnostic ultrasound device 14 to be placed with the VGA display screen down, such that the key-pad is exposed for one button access of diagnostic ultrasound device 14 features.

FIG. 5 shows an alternate use of the present invention. The mechanical sector probe 74, which is connected to the diagnostic ultrasound device 14 by a one-meter probe cable 60, is held against the neck to look for fluid associated with a neck injury. The sonographer holds the diagnostic ultrasound device 14 in her hand to observe and record the image as it appears on the LCD display 18 with audio tags. Images from previous visits can be retrieved by wireless-link or pre-loaded by USB (or even a flash card assigned to each patient) to the LCD display 18 for diagnosing the progress of the injury. These images can be sent via e-mail to a radiologist for additional comments and post processing to develop a therapeutic plan.

FIG. 6 shows an alternate use of the present invention. The diagnostic ultrasound device 14 is in a carry pouch 72 on the belt of the EMS technician. The technician can use the linear array ultrasound probe 75 to look for metal embedded deep into the body from an auto accident or with a phased array probe that has Doppler capability, listen for heartbeat in a noisy environment. The heartbeat is detected and read out on the HMD. The condition of the victim can be audio tagged to the frozen spectrogram that shows the heartbeat. The EMS technician can use the probe to look for neck contusions or fluid that may appear around distressed fractures before the victim is moved. The electret condenser microphone 70 can capture information from the victim as he regains consciousness such as accident details or description of the event. The exact ratio of audio recorded is what the EMS technician hears in the HMD ear speaker 68.

FIG. 7 shows detail of the head mounted display 64 (HMD) glasses. The HMD upper case 76 is made of a molded nylon material wherein the audio and microphone levels are mixed by an Audio/Microphone Balance Control 80 on the top of the HMD. The HMD glasses are kept in position on the operator's head by use of elastic HMD head strap 83, such that they do not fall off when the operator bends down. The HMD glasses adjust to the physical requirements of the viewer by an HMD ear-speaker position mechanism 84 that allows the ear speaker 68 to be positioned and locked in place. The HMD glasses may be folded at the HMD hinge 88 such that they can be put in a shirt pocket. The HMD display housing holds the HMD backlight 90 and power supply on a small circuit board and two small HMD VGA displays 92. Such displays are typified by the Kopin™ 640C VGA display, which is a low power micro-display as would be used in a camera viewfinder. The HMD VGA displays 92 (0.7″ or smaller) are positioned in the HMD Upper Case 76 such that the HMD Backlight 90 is in a horizontal plane facing downward to be reflected off a surface HMD mirror 94 or optical prism and directed toward the HMD lens 96. These HMD mirror 94 and HMD lens 96 are focused to present a binaural image to the sonographer. A small circuit board (not shown) interfaces the optics and provides composite video decoding and sync signals for the LCD display 18 NTSC video signals. The HMD Lower Case 98 is clear to allow the lower ⅓ of vision so that the sonographer can see to interact with patient. The clear acrylic type material wraps around the user's face to offer protection from flying debris and balance the HMD design.

FIG. 8 shows a block diagram of the diagnostic ultrasound device 14 in accordance with the present embodiment. The printed circuit board 100 and the components contained thereupon will be discussed in detail. It should be apparent to those skilled in the art that components will be improved or discontinued over time and this does not compromise the intent of the present embodiment of the diagnostic ultrasound device 14. 64 or 128 transducers are connected to high voltage mux 102 integrated circuits such as those manufactured by Supertex™ (HV230TA). The high voltage mux 102 board also contains an RFID transceiver (not shown) made by Texas Instruments TIRIS™ division for 134 Hz RFID tag reading and writing for veterinary applications. This probe board is connected by probe cable 60 and probe connector 30 to the single printed circuit board 100 that houses the control electronics for the diagnostic ultrasound device 14 of the present invention.

Probe geometry and the required timing drives the setup of the beamformer. For instance, the mechanical probes have one crystal and are driven by a motor such as those manufactured by Micro Mo™ that offer 512 optoisolator pulses per turn of the crank with one index pulse at the sector perpendicular location. This means that addressing is symmetrical about Theta=0 degrees. Typically, there will be 120 vectors at 0.75 degree (90 degrees total) that contain 304 samples in RHO for each acoustic line formed. To acquire a full frame of 18 cm field of view, it requires:
1.3 us/mm*180 mm*120 lines=27.6 ms

This is under the required 30 frames per second of a “real time” diagnostic ultrasound device 14. However, this frame rate is not sufficient for cardiology. Using a curved array probe 58, the frame rate can be increased by using a synthetic transmit aperture. (Holm) The subaperture transmits (1 . . . 32 xtals) to an area of interest in the tissue and then the received echoes are dynamically focused for the entire aperture. For example, if a 128-element crystal array probe is used; four sub-apertures can be selected to reduce the channel count to 32. Simultaneous non-overlapping subapertures allow an increase in the frame rate, as the normal acquisition time for the ultrasonic pulses would be divided by the number of subapertures used. Typically, there will be eight or less subapertures or zones for the entire field of view.

A printed circuit board 100 contains the primary system components of the diagnostic ultrasound device 14. The high voltage mux 102 outputs are electrically connected to an octal vca 106 (voltage controlled amplifier) such as those manufactured by Texas Instruments (VCA8613) where the very small (100 mv) ultrasonic ring is processed by +26 db LNA, TGC time gain compensation) and programmable gain stage. The octal vca 106 TGC analog input allows gain to be increased as a function of distance in the tissue to compensate for attenuation. The octal vca 106 gain stage is serially programmable in 4 db steps to a maximum of 40 db. The TGC ramp is produced by an 8-bit TGC D/A 108 (digital to analog converter) that is driven by lookup tables in the field programmable gate array. The differential vca outputs are sent to a low voltage, 40 MSPS 10-bit octal A/D 110 (analog to digital converter). This device is typically a Texas Instruments AD5121. If all channels of the octal A/D 110 are not used, they may be powered down, as would be the case for mechanical sector probe 74. The Field Programmable Gate Array (FPGA 112) clocks all channels simultaneously to preserve the RF phase information. The digitized raw ultrasound data is stored in the RF frame memory 113. The samples are converted to I (In Phase) and Q (Quadrature) terms by baseband demodulation 114. (Tomov) The 16-bit words (8-bit Magnitude and 8-bit Phase) are match filtered then forwarded to the beamformer section of the FPGA 112. To accomplish these signal processing functions, an FPGA 112 is selected that is low cost, low power and offers adequate embedded_sram 138 memory. A suitable FPGA 112 is available from Xilinx™ in the Virtex2 Pro™ series. The Virtex2 Pro™ device contains a PowerPC™ 405 microprocessor. This microprocessor can perform the high speed, parallel data processing at 400 DMIPS. Alternative means of implementing the beamformer could be a state machine coded in HDL.

At least two RF Frame Memory 113 are possible in the FPGA 112 device. One of the RF Frame Memory 113 stores the new data while the other RF Frame Memory 113 is processing the previous data. The RF data may be frame averaged and stored back to the incoming RF Frame Memory 113 buffer. This data is beamformed and focused by the FPGA 112 at the raster output pixel position (Pixel Point Beamformer™) as will be discussed in great detail. After beamforming and scan conversion, data is dynamically band pass filtered depending on the frequency of the probe (f0) and the sample depth.

Inside the FPGA 112 the beamformed N-bit I and Q terms are peak detected and used as an index into a log compression table to limit the dynamic range to a range suitable for the LCD display 18 device. (Tomov) The log compression table also gamma corrects the pixel for the LCD display 18 device. The processed pixel frame is stored by the dual-core processor 116 DMA (Dynamic Memory Access) to SDRAM 117 (Synchronous Dynamic Random Access Memory). The SDRAM 117 may be used for frame averaging 2-4 video frames prior to transfer to the LCD display 18 buffer. The dual-core processor 116 is part of the Texas Instruments OMAP™ (Open Multimedia Application Platform) family. The dual-core processor 116 will be further explained in detail.

The storage media that the dual-core processor 116 uses for the video frame buffer is typically a 128 MB MOBILE DDR Dual Data Rate) SDRAM 117. A Disk On Chip 118 TM device is used that offers 32 megabytes of NAND FLASH. This device is typically manufactured by M-Systems™. This device is used for programming the FPGA 112 and other tables for the diagnostic ultrasound device 14 of the present invention. The Disk On Chip 118 TM allows operating systems such as embedded Linux or Windows CE™ to run on the dual-core processor 116. The Boot Device (not shown) is typically a 4 MB NOR flash device as manufactured by AMD™.

A keypad and trackball 119 device is connected to the dual-core processor 116. A wireless link 120, which may utilize an encrypted 802.11B/G protocol, may be used to transmit diagnostic ultrasound image files to a local WLAN inside the hospital or clinic. The wireless link 120 may offer Bluetooth™ protocol at reduced computational overhead to the dual-core processor 116.

The octal vca 106 also provides a CW (continuous wave) matrix of the LNA outputs that may be current summed to drive the input of an analog delay line 122. In this fashion, the CW matrix may use half or selected portions of the crystal array to focus the ultrasonic beam (beamforming) as is well known to those familiar with the art. This focused CW area of interest is user positioned by the trackball 22 device. The samples are first analog filtered by a four-pole high pass filter 123 to remove “wall” reflections from stationary walls and slow moving tissue. The samples are then converted to differential mode for input to a fast S/H (Sample and Hold) 16-bit A/D 124 such as the ADS1611 manufactured by Texas Instruments™. The A/D outputs a 2V-centered word in 2's compliment format with fast settling time up to the natural 10 MHZ limit of the ADS1611 Delta-Sigma typology. Doppler probe frequency will be in the 2 MHZ-4 MHZ band. These samples are then baseband I and Q demodulated in the FPGA 112. The process of autocorrelation is performed in the FPGA 112. An FFT is simultaneously performed by the dual-core processor 116 embedded digital signal processor. (Jensen and Kasai)

A graphics processor 126 may be used to convert the (320×240×16 bpp) to (640×480×16 bpp) for the high resolution LCD Display 18. Such a display is typified by the Toshiba VGA display LTM04C380K that is a low power p-Si 4″ TFT LCD Display 18. This scaling and pixel clock increase could be done in the FPGA 112 or a dedicated graphics co-processor as manufactured by NVIDIA (MediaQ™) and ATI (IMAGEON™). Frame conversion and synchronization to NTSC specifications may also be performed at this point. The NTSC standard is required for video display on the HMD. Some of the graphic processors offer hardware JPEG encode and decode or it can be performed by the digital signal processor in dual-port on-chip memory. Once the image is JPEG encoded, it can be stored on the removable secure digital flash card 26 and can be accessed by a host pc over the USB. The image may also be transferred by wireless link 120 to a local area network (LAN) for storage with the patient record.

A power supply and battery charger 130 converts the battery voltage into all system operating voltages. The battery charger monitors the complete state of the battery and the status of the battery is shown on the LCD display 18 and led 24 as previously discussed. Such a suitable battery charging IC is available from Maxim™ as MAX1758. This battery charging IC will fully charge the system battery in approximately 3 hours. The battery voltage is converted to system voltages by a switch mode power supply controller. Such a suitable controller is available from Maxim™ as MAX1774. This controller provides 2 A of current and with a single external mosfet. The controller also provides a battery switch and detection of the external 9V DC source.

FIG. 9 is a block diagram of a typical dual-core processor 116 and the functions used by the ultrasound device. It should be understood by those familiar in the art that this is a typical dual-core processor 116, but that this technology is subject to change that will not affect the intent of the use of such technology for the diagnostic ultrasound device 14. The dual-core nature of this processor allows a low power, integrated design that allows many of the functions of the diagnostic ultrasound device 14 to be changed in software, and allows all of the functions of the diagnostic ultrasound device 14 to be programmed through USB and other ports, as they exist on the dual-core processor 116. Key functions of the dual-core processor 116 are the multiple levels of DMA (Dynamic Memory Access) and multiple threads available on chip for communication with memory and peripherals. This allows low power inexpensive memory to be shared and tasks to be scheduled and prioritized as needed by the system.

The dual-core processor 116 will now be discussed in detail and specifically, the advantages gained from its use in the present embodiment of the diagnostic ultrasound device 14. The MPU 132 (Microprocessor Unit) has a unified address space. Therefore, the internal and external memories for program and data as well as peripheral registers and configuration registers are all accessed within the same address space. The MPU 132 space is always addressed using byte addressing. The MPU 132 accesses peripheral and configuration registers in the same way that internal and external memory is accessed. The DSP 134 (Digital Signal Processor) subsystem contains 160 K bytes of on-chip SRAM 138 64 K bytes of DARAM and 96 K bytes of SARAM). The DSP 134 also has access to the shared system SRAM 138 2 M bit and both EMIF (External Memory Interface) spaces EMIFF (fast) and EMIFS (slow) via the DSP 134 Memory Management Unit (MMU), which is configured by the MPU 132. In the DSP 134, the DARAM is composed of eight blocks of 8 K bytes each. Each DARAM block can perform two accesses per cycle (two reads, two writes, or a read and a write). The SARAM is composed of 12 blocks of 8 K bytes each. Each SARAM block can perform one access per cycle (one read or one write). The DSP 134 can operate on its own DARAM and SARAM so that for instance, an FFT (Fast Fourier Transform) can be performed on an ensemble of Doppler samples while the MMU is streaming live diagnostic ultrasound images to the LCD Display 18.

The DSP 134 I/O space is a separate address space from the data/program memory space. The I/O space is accessed via the DSP's port instructions. The DSP 134 I/O space is accessed using 16-bit word addresses. The memory interface traffic controller 140 allows memory access by the DSP MMU 146. The DSP MMU 146 is controlled by the MPU 132. The MPU 132 and DSP 134 each have their own separate private peripheral bus. Their respective processors may only access peripherals on each of these private buses. For instance, the DSP 134 timers on the DSP private peripheral 148 bus are not accessible by the MPU private peripheral 150 bus.

The Real-Time Clock 152 (RTC) module provides an embedded RTC for use in applications that need to track real time like the time and date display on the LCD. This peripheral is an ultra-low-power module meaning that the RTC module can be powered independently without powering the dual-core processor 116 MPU 132 core.

The LCD controller 154 is configured by the MPU 132 and utilizes a dedicated channel on the System DMA controller 156 to transfer data from the frame buffer. The frame buffer can be implemented using the internal shared SRAM 138 (Static Random Access Memory) (2 M bit) or optionally using external SDRAM 117 via the EMIFF. Using the frame buffer as its data source, the System DMA must provide data to the FIFO at the front end of the LCD display 18 controller data path at a rate sufficient to support the chosen display mode and resolution. Optimal performance is achieved when using the internal SRAM 138 as the frame buffer. The LCD panel size is programmable, and can be any width (line length) from 16 to 1024 pixels in 16-pixel increments. Programming the total number of pixels in the LCD display 18 sets the number of lines. The frame buffer required for the diagnostic ultrasound device 14 is 320×240×16-bit (1,228,800 bits). A graphics processor 126 or the FPGA 112 will pixel-double the image to 640×480×16-bit (4,915,200 bits) for VGA on the LCD display 18 and NTSC head mounted display 64.

The MPU 132 and the System DMA controller 156, which is configured by the MPU 132, may only access peripherals via the MPU Bridge 157 if they are on the MPU Public 158 Peripheral bus. The system DMA controller 156 can access MPU Public 158 Peripheral bus by a local bus 160. The DSP 134 cannot access peripherals on this bus. The dual-core processor 116 USB host controller communicates with USB devices at the USB low-speed (1.5 M-bit/s maximum) and full-speed (12 M-bit/s maximum) data rates. The USB Function emulates a disk storage device such that ultrasound images on the removable Secure Digital Flash card 26 can be accessed from a personal computer system.

The MMC/SD (multimedia media/secure digital) Interface controller, on the MPU Public 158 Peripheral bus, provides an interface to MMC or SD memory cards plus up to three serial SPI flash cards. The controller handles MMC/SD or SPI transactions with minimal MPU 132 intervention, allowing optional use of two System DMA controller 156 channels for transfer of data. The MPU 132 software must manage transaction semantics, while the MMC/SD controller deals with MMC/SD protocol at the transmission level: packing data, adding the CRC, generating the start/end bit and checking for syntactical correctness. When interfacing with 8-bit SPI devices, the MMC/SD module does not perform any MMC specific function; rather it provides a generic SPI interface. Several additional interface pins are utilized to provide the SPI clock and SPI chip selects. HDQ and 1-Wire interfaces are available for battery management and power management of the diagnostic ultrasound device 14. The interface can be used to send command and status information between dual-core processor 116 and the Li-Ion Battery 46.

The CAMERA interface is an 8-bit external port on the MPU Public 158 Peripheral bus, which may be used to accept data from the Pixel Point Beamformer™ in the FPGA 112. The interface handles multiple image formats synchronized on vertical and horizontal synchronization signals. Data transfer to the camera interface may be done synchronously or asynchronously. The camera interface module converts the 8-bit data transfers into 32-bit words and utilizes a 128-word buffer to facilitate efficient data transfer to memory. Data may be transferred from the camera interface buffer to internal memory by the System DMA controller 156 or directly by the MPU 132. The interface may utilize an externally driven clock at rates up to 13 MHz or may optionally provide an output reference clock at rates of 8 MHz, 9.6 MHz, or 24 MHz when the camera interface is configured for clocking from the internal 48 MHz clock. The CAMERA interface is the bus by which byte data from the FPGA 112 that contains the beamformed I/Q samples (depending on mode of operation) will arrive. The automatic packing of these bytes facilitates efficient memory transfer to storage or other processing.

The MPUIO pins may be used as either general-purpose I/O for the MPU 132 or as a Keyboard Interface to a 4×4 keypad 20. Since a 4×4 keypad 20 array is implemented, the unused MPUIO pins are used as GPIO. When used as GPIO, each pin may be configured individually as either an output or an input, and they may be individually configured to generate MPU 132 interrupts based on a level change (falling or rising) after a debouncing process. These MPUIO interrupts may be used to wake up the device from deep-sleep mode using the 32-kHz clock. The MPUIO pins may also be used as a keyboard interface. In the preferred embodiment of the diagnostic ultrasound device 14, these GPIO pins are used for Motor Position Opto-Isolator Pulses and externally buffered probe function keys, for instance “FREEZE”.

The Pulse-Width Light (PWL) module provides control of the LCD display 18 or keypad 20 backlighting by employing a random sequence generator. This voltage-level control technique decreases the spectral power at the modulator harmonic frequencies. This PWL would modulate the CCFL or LED 24 backlight source as manufactured by Tamura and others. The module uses a switchable 32-kHz clock. The Pulse-Width Tone PWT) module generates a modulated frequency signal for use with a buzzer. There are two separate LED 24 Pulse Generator LPG) modules. Each LPG module provides an output for an indication LED 24. The blinking period is programmable between 152 ms and 4 s or the LED 24 can be switched on or off permanently. The LED 24 Pulse generators each control one third of the system LED 24 (Orange and Green). The Blue LED 24 is connected to the main system power rail.

The Multichannel Buffered Serial Port (McBSP) provides a high-speed, full-duplex serial port that allows direct interface an audio codec (TLV320AIC23) that feeds audio to the HMD ear speaker 68. McBSP2 is used for an 8 channel A/D (TLV1570) that monitors battery, system voltages and probe voltages like ID and Backlash from the motor for mechanical sector probes. The 12C port is used to control a Chrontel CH7013B PC-NTSC converter that supplies the composite video to the HMD glasses. The CH7013 is used in 5-6-5 non-multiplexed mode at 24.671 MHz. This is the common format and pixel clock for both the NTSC converter and the LCD display 18 panel. A graphics processor 126 or FPGA 112 may be used to achieve this pixel rate after processing the LCD controller 154 output.

The Multichannel Serial Interface (MCSI) provides flexible serial interface with multichannel transmission capability. The MCSI allows the DSP 134 to access a variety of external devices, such as audio codecs and other types of analog converters. The diagnostic ultrasound device 14 of the present invention uses these MCSI ports to load registers in the VCA's that are used to set the Gain and Doppler CW Matrix.

The MPU/DSP Shared Peripherals 164 are connected to both the MPU Public 158 Peripheral bus and the DSP Public 161 Peripheral bus. In the case of the UARTs, these connections are achieved via a Tl Peripheral Bus Switch, which must be configured to allow MPU 132 or DSP 134 access to the UARTs. As an example, the UART1 could be used for wireless interface to a Taiyo Yuden™ BRF6100 based Bluetooth™ module that emulates a wireless serial connection to the diagnostic ultrasound device 14. The McBSP3 or EMIFF may be used to connect to a TNET1100B based module manufactured by National Datacomm Corporation for wireless 802.11b/g WLAN connection. Such a connection could transfer the RFID data, Scan Data or Patient Data to a local network. An image could be retrieved or sent to the WLAN network installed in many hospitals and clinics.

There are 14 or more shared GPIO pins on the dual-core processor 116 device, which may be accessed and controlled by either the DSP public 161 peripheral bus or the MPU public 158 peripheral bus. Each GPIO pin is independently configurable to be used by either the DSP 134 or MPU 132. The MPU 132 controls which processor owns each GPIO pin by configuring a pin control register that only the MPU 132 can access. Each GPIO pin can be used as either an input or output pin with GPIO inputs being synchronized internally to a peripheral clock. GPIO inputs may also optionally be configured to generate an interrupt condition to the processor that owns the GPIO pin. The sense of the interrupt condition is configurable such that either a high-to-low or low-to-high transition causes the interrupt condition. Some of the GPIO pins are multiplexed with other interface pins specific to other device peripherals.

The System Direct Memory Access (DMA) controller transfers data between points in the memory space without intervention by the MPU 132. The System DMA controller 156 allows movements of data to and from internal memory, external memory, and peripherals to occur in the background of MPU 132 operation. It is designed to off-load the block data transfer function from the MPU 132 processor. The System DMA controller 156 is configured by the MPU 132 via the MPU private peripheral 150 bus. The System DMA controller 156 has at least nine independent general-purpose channels and seven ports that it may transfer to/from. An additional channel is dedicated for use with the LCD display 18 controller. Of the many available ports, the DMA transfers may occur between any two ports with the exception of the LCD display 18 port, which may only be used as a destination with the EMIFF or IMIF as the source. For maximum transfer efficiency, all DMA channels are independent. This means that if multiple channels are exclusively accessing different ports, then simultaneous transfers performed by the channels will occur uninhibited. If the multiple channels are accessing common ports, however, some arbitration cycles will be necessary. Arbitration occurs in a round-robin fashion with configurable priority for each channel (high or low).

The DSP 134 subsystem has its own dedicated DMA Controller, which is entirely independent of the MPU 132 or the System DMA controller 156. The DSP 134 DMA Controller has many of the same major features that the System DMA controller 156 possesses. The DSP 134 DMA Controller has six generic channels and five physical ports available for source or destination data. These five ports are the SARAM port, DARAM port, EMIF (External memory port), DSP 134 TIPB port, and MPUI port. The DSP 134 may configure the DSP 134 DMA Controller to transfer data between the SARAM, DARAM, EMIF, and TIPB ports, but the MPUI port is a dedicated port used for MPU 132 or System DMA controller 156 initiated transfers to DSP 134 subsystem resources. The SARAM and DARAM ports are used to access local DSP 134 memories and the TIPB port is used to access the registers of the DSP 134 peripherals. The EMIF port of the DSP 134 DMA controller is used to access the Traffic Controller via the DSP MMU 146 (Memory Management Unit).

The Memory Interface Traffic Controller 140 (TC) manages all accesses by the MPU 132, DSP 134, System DMA controller 156, and Local Bus 160 to the dual-core processor 116 system memory resources. The EMIFF Interface provides access to 16-bit-wide SDRAM 117 memories and the IMIF provides access to the 2 M bit of on-chip SRAM 138. The TC provides the functions of arbitrating contending accesses to the same Memory Interface Traffic Controller 140 from different initiators MPU 132, DSP 134, System DMA controller 156, and Local Bus 160), synchronization of accesses due to the initiators and the memory interfaces running at different clock rates, and the buffering of data allowing burst access for more efficient multiplexing of transfers from multiple initiators to the memory interfaces. The TC's architecture allows simultaneous transfers between initiators and different memory interfaces without penalty. For instance, if the MPU 132 is accessing the EMIFF at the same time, the DSP 134 is accessing the IMIF, transfers may occur simultaneously since there is no contention for resources. There are three separate ports to the TC from the System DMA controller 156 one for each of the memory interfaces), allowing for greater bandwidth capability between the System DMA controller 156 and the TC. Several mechanisms allow for communication between the MPU 132 and the DSP 134 on the dual-core processor 116 device. These include mailbox registers, MPU Interface 162, and shared memory space. The MPU 132 and DSP 134 processors may communicate with each other via a mailbox-interrupt mechanism. This mechanism provides a very flexible software protocol between the processors.

The MPU interface 162 (MPUI) allows the MPU 132 and the System DMA controller 156 to communicate with the DSP 134 and its peripherals. The MPUI allows access to the full memory space (16 M bytes) of the DSP 134 and the DSP public 161 peripheral bus. Thus, the MPU 132 and System DMA controller 156 both have read and write access to the complete DSP 134 I/O space (128 K bytes), including the control register of the DSP public 161 peripherals. The dual-core processor 116 device implements shared memory architecture via the Traffic Controller. Therefore, the MPU 132 and DSP 134 both have access to the same shared SRAM 138 memory (2 M bit) as well as to the EMIFF and EMIFS memory space.

The TMS320C55x DSP 134 core within the dual-core processor 116 device utilizes three powerful hardware accelerator modules, which assist the DSP 134 core in implementing specific algorithms that are commonly used in video compression applications such as MPEG4 encoders/decoders. These accelerators allow implementation of such algorithms using fewer DSP 134 instruction cycles and dissipating less power than implementations using only the DSP 134 core. The hardware accelerators are utilized via functions from the TMS320C55x Image/Video Processing Library available from Texas Instruments™. The DCT/iDCT hardware accelerator is used to implement Forward and Inverse DCT (Discrete Cosine Transform) algorithms. These DCT/iDCT algorithms can be used to implement a wide range of video compression standards including JPEG Encode/Decode, MPEG Video Encode/Decode, and H.26x Encode/Decode. The Motion Estimation hardware accelerator implements a high-performance motion estimation algorithm, enabling MPEG Video encoder or H.26x encoder applications. Motion estimation is typically one of the most computation-intensive operations in video-encoding systems. The Pixel Interpolation Accelerator enables high-performance pixel-interpolation algorithms. Such algorithms provide significant improvement to video-encoding applications. The diagnostic ultrasound device 14 uses the DCT/iDCT for image encode/decode to/from the secure digital flash card 26. The MPEG function is used for the Cineloop™ storage and the motion estimation allows regional pixel interpolation to be performed such that the image is compensated for movement of the diagnostic ultrasound probe 75. A JTAG Test 168 port is available for emulator debug of the dual-core processor 116 subsystem.

To those familiar in the art it is well understood that the specifications and speed of such a dual-core processor 116 are likely to improve and those improvements can be easily incorporated into the diagnostic ultrasound device 14 as they become available. The tightly coupled design of the ARM™ MMU and TI DSP 134 on one silicone die makes possible levels of hardware effectiveness that is difficult to achieve with a non-integrated solution.

FIG. 10 describes the steps of the preferred embodiment of the Pixel Point Beamformer™. For the diagnostic ultrasound of the present invention, the FPGA 112 allows beamforming to be done on the raw data samples at the P(x, y) pixel coordinates required by the current display mode. Data is filtered, frame averaged and converted to (I/Q) pairs in the FPGA 112 and stored to the RF Frame memory 113. Data is recalled by the FPGA 112 based on the required P(x, y) pixel of interest. In general, a pixel that represents the Pixel Point of interest plus the surrounding four pixels to the Pixel Point of interest will be used for interpolation. The Pixel Point of interest has a calculated distance based on time of flight from any transducer element and the speed of sound approximately 1.3 us/mm round trip. The pixel must be tested to see if it is in the area of interest (Is Pixel in Display Region? 180). If it is, we calculate distance×sound velocity=time index 182. When a valid x location is found, the four closest samples 184 “in time” are fetched from memory and are interpolated (weighting factors for these samples 186). The contributions of the four closest samples 184 are calculated by the weighting factor times the intensity value of the contributing pixel. This gives the intensity value of the pixel of interest 188, however this must be repeated for all N transducers that contribute to the Pixel Point of interest to focus the pixel until we are Done N? 190; Else (N++192). When all (I/Q) contributions from all N transducers for the pixel of interest are made the summed, (I/Q) samples are detected as:
P(x,y)=Σ\sqrt{I²+Q²}

This result is translated by a log lookup and gamma correction table 194 and stored as an 8-bit pixel intensity value in the DDR SDRAM 117 memory of the dual-core processor 116. The process continues in horizontal raster fashion until we are Done X? 196; Else increment X 198 to the end of the raster line. Then, X is reset to “0” and we continue in vertical raster fashion until we are Done Y? 200; Else increment Y 202 until we reach the End of Frame 204. This process continues until all of the valid pixels are processed for a complete frame of raster video. The completed data frame in raster format is transferred to the dual-core processor 116 via the camera interface port. The camera interface port accepts 8-bit (byte) transfers with a synchronous transfer clock up to 24 MHz. The frame is sent to the dual-core processor 116 video buffer for output to the LCD controller 154 or HMD. The process described above can occur in the FPGA 112 with a small embedded microprocessor core such as the Xilinx Microblaze™ or PowerPC™ controlling the Pixel Point Beamformer™.

FIG. 11 is a subsystem block diagram showing the power supply and battery charger 130 circuitry of the diagnostic ultrasound of the present invention. An external 9V DC wall cube 218 source @ 15 watts provides the diagnostic ultrasound device 14 with power for charging the Li-Ion Battery 46 pack. The Li-Ion charger 222 monitors temperature of the Li-Ion Battery 46 pack by a thermistor and controls the charging profile of the Li-Ion Battery 46 pack. Maxim (MAX1758) makes a suitable battery charger IC. The Li-Ion Battery 46 pack has two series groups of Li-Ion cells in parallel for a combined Li-Ion Battery 46 pack capacity of 8.4 volts @ 4000 mAh. This will run the diagnostic ultrasound device 14 of the current invention for four hours or more depending on probe and mode of use. The Li-Ion Battery 46 pack is removable so that a spare battery can be used at any time. The Li-Ion Battery 46 supplies a high voltage power supply 224 that generates the +100 and −100 voltage rails for linear and sector probe transmits. These are low current rails in the low milliamp range. The dual-core processor 116 monitors all diagnostic ultrasound functions and issues power down to those not currently in use to achieve a long runtime with the Li-Ion Battery 46. In the preferred embodiment, an integrated DC-DC controller as typified by Maxim MAX1774 allows the 9V DC Wall Cube 218 to be detected and used if connected to the diagnostic ultrasound device 14. If the 9V DC Wall Cube 218 is not present, a low rds-on FET connects the Li-Ion Battery 46 pack to the DC-DC controller. This controller provides two regulated voltage rails and two of these controllers are used to generate the DC-DC Controller (5V/1.8V) 226 and DC-DC Controller (3.3V/1.2V) 228 rails for the diagnostic ultrasound device 14. An additional LDO regulator provides auxiliary voltage of LDO (2.5V) 230. The LDO (1.6V) 232 operates directly from the Li-Ion Battery 46 pack and supplies a keep-alive battery 234 (1.6V) for the dual-core processor 116. This means that the dual-core processor 116 is always powered even though it may be in sleep state to conserve battery until needed. It is well understood by those skilled in the art that other power supply schemes might allow similar functionality for the diagnostic ultrasound device 14 of the present invention.

FIG. 12 shows the Doppler processor 238 diagram. The octal vca 106 has a selectable CW matrix output of all channels. These outputs are current summed so they can be added to various taps of an analog delay line 122. This analog delay line 122 is for beamforming and will be well known to those familiar with the art. This allows the user to steer the CW receive delays to allow a region of interest to be interrogated. This also allows receive selection from various angles of incidence to the transducer surface for improved Doppler accuracy. The selected, focused and summed RF is then processed by a high pass filter 123 to remove the “wall” artifacts below 40 Hz. This allows increased sensitivity at the S/H 16-Bit A/D 124. The high pass filter 123 also converts the single ended RF to differential RF so that the differential S/H 16-Bit A/D 124 can process the signal with high SNR (signal to noise ratio). Such a converter is manufactured by Texas Instruments as ADS1611. As an example, a 3 MHz RF is first mixed with a synthesized 2 MHz. This produces 5 MHz and 1 MHz IF frequency components. The IF is mixed with 1 MHz 0 degrees and 1 MHz 90 degrees (Quadrature Demodulator 240) and matched band pass filter 250 to produce (I/Q) baseband. These I and Q signals are matched band pass filtered to remove the 1 MHz mixing signal and band limited to the 40 Hz to 15 kHz Doppler range. This complex (I/Q) word is processed by a Hilbert 260 Transform and Delay 262 for audio output of the CW Doppler flow 264 signal that is CODEC processed and amplified for the ear speaker 68 on the HMD.

The I/Q sample pairs are stored in the FPGA 112 by a line FIFO 252 so that a compare 254 of the previous transmit samples by an auto-correlator 256 over one transmit is possible. (Jensen) This correlated difference is processed by a peak detector 258 and sent to the dual-core processor 116. A complex FFT 266 is simultaneously performed by the dual-core processor 116 on the same I/Q samples to determine the frequency shift of the Doppler flow 264 and its movement over time. A complex FFT 266 frequency group is then forwarded to the velocity and variance estimator 268 where the Doppler data is processed by a priority encoder 270 to determine if it should replace a grey scale pixel in the region of interest. If so, the color lookup table 272 will turn the Doppler pixel value into appropriate shades of blue showing Doppler flow 264 away from the viewer or red showing Doppler flow 264 toward the viewer. The shade of blue or red will be dependent on the velocity of blood as is well known to those familiar with the art. This pixel color overlay will be placed in the RGB Video Buffer 274 for LCD display 18.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

1. A handheld diagnostic ultrasound system with head mounted display for ultrasound imaging allowing hands free operation comprising:
means for processing the diagnostic ultrasound image;
means for displaying said diagnostic ultrasound image;
means for storing multiple diagnostic ultrasound images;
means for powering the diagnostic ultrasound device;
means for cable transfer of the diagnostic ultrasound image to pc or other storage;
means for viewing the diagnostic ultrasound image in adverse lighting conditions;
means for interrogating the tissue in the region of interest, electrically connected to said means for processing the diagnostic ultrasound image;
means for beamforming the diagnostic ultrasound image, electrically connected to said means for processing the diagnostic ultrasound image;
means for storing at least one frame of rf data, electrically embedded to said means for beamforming the diagnostic ultrasound image;
means for seperating the incoming rf samples into i (in-phase) and q (quadrature) componets, electrically embedded to said means for beamforming the diagnostic ultrasound image;
means for program and data storage of the dual-core processor;
means for storing operating code of the dual core processor;
means for operator interface;
means for wireless transfer of the ultrasound image to pc or other storage;
means for converting lcd signals into composite ntsc video signals, electrically connected to said means for viewing the diagnostic ultrasound image in adverse lighting conditions; and
means for processing the ultrasound image for lcd display.

2. The handheld diagnostic ultrasound system with head mounted display in accordance with claim 1, wherein said means for processing the diagnostic ultrasound image comprises a handheld, PDA style diagnostic ultrasound device.

3. The handheld diagnostic ultrasound system with head mounted display in accordance with claim 1, wherein said means for displaying said diagnostic ultrasound image comprises a lcd display.

4. The handheld diagnostic ultrasound system with head mounted display in accordance with claim 1, wherein said means for storing multiple diagnostic ultrasound images comprises a non-volatile secure digital flash card.

5. The handheld diagnostic ultrasound system with head mounted display in accordance with claim 1, wherein said means for powering the diagnostic ultrasound device comprises a li-ion battery.

6. The handheld diagnostic ultrasound system with head mounted display in accordance with claim 1, wherein said means for cable transfer of the diagnostic ultrasound image to pc or other storage comprises an usb connection.

7. The handheld diagnostic ultrasound system with head mounted display in accordance with claim 1, wherein said means for viewing the diagnostic ultrasound image in adverse lighting conditions comprises a head mounted display, having a wide field of view and audio ear speakers.

8. The handheld diagnostic ultrasound system with head mounted display in accordance with claim 1, wherein said means for interrogating the tissue in the region of interest comprises an ultrasound probe.

9. The handheld diagnostic ultrasound system with head mounted display in accordance with claim 1, wherein said means for beamforming the diagnostic ultrasound image comprises a low power, low voltage fpga.

10. The handheld diagnostic ultrasound system with head mounted display in accordance with claim 1, wherein said means for storing at least one frame of rf data comprises a rf frame memory.

11. The handheld diagnostic ultrasound system with head mounted display in accordance with claim 1, wherein said means for seperating the incoming rf samples into i (in-phase) and q (quadrature) componets comprises a baseband demodulation.

12. The handheld diagnostic ultrasound system with head mounted display in accordance with claim 1, wherein said means for program and data storage of the dual-core processor comprises a low power, low voltage, DDR sdram.

13. The handheld diagnostic ultrasound system with head mounted display in accordance with claim 1, wherein said means for storing operating code of the dual core processor comprises a low power, low voltage, non-volatile disk on chip.

14. The handheld diagnostic ultrasound system with head mounted display in accordance with claim 1, wherein said means for operator interface comprises a keypad and trackball.

15. The handheld diagnostic ultrasound system with head mounted display in accordance with claim 1, wherein said means for wireless transfer of the ultrasound image to pc or other storage comprises a wireless link.

16. The handheld diagnostic ultrasound system with head mounted display in accordance with claim 1, wherein said means for converting lcd signals into composite ntsc video signals comprises a graphics processor.

17. The handheld diagnostic ultrasound system with head mounted display in accordance with claim 1, wherein said means for processing the ultrasound image for lcd display comprises a lcd controller.

18. A handheld diagnostic ultrasound system with head mounted display for ultrasound imaging allowing hands free operation comprising:
a handheld, PDA style diagnostic ultrasound device, for processing the diagnostic ultrasound image;
a lcd display, for displaying said diagnostic ultrasound image;
a non-volatile secure digital flash card, for storing multiple diagnostic ultrasound images;
a li-ion battery, for powering the diagnostic ultrasound device;
an usb connection, for cable transfer of the diagnostic ultrasound image to pc or other storage;
a head mounted display, having a wide field of view and audio ear speakers, for viewing the diagnostic ultrasound image in adverse lighting conditions;
an ultrasound probe, for interrogating the tissue in the region of interest, electrically connected to said diagnostic ultrasound device;
a low power, low voltage fpga, for beamforming the diagnostic ultrasound image, electrically connected to said diagnostic ultrasound device;
a rf frame memory, for storing at least one frame of rf data, electrically embedded to said FPGA;
a baseband demodulation, for seperating the incoming rf samples into i (in-phase) and q (quadrature) componets, electrically embedded to said FPGA;
a low power, low voltage, DDR sdram, for program and data storage of the dual-core processor;
a low power, low voltage, non-volatile disk on chip, for storing operating code of the dual core processor;
a keypad and trackball, for operator interface;
a wireless link, for wireless transfer of the ultrasound image to pc or other storage;
a graphics processor, for converting lcd signals into composite ntsc video signals, electrically connected to said head mounted display; and
a lcd controller, for processing the ultrasound image for lcd display.

19. The handheld diagnostic ultrasound system with head mounted display as recited in claim 18, further comprising:
a low power, highly integrated dual-core processor, for control and processing of all diagnsotic ultrasound images, electrically connected to said diagnostic ultrasound device, electrically connected to said LCD Display, electrically connected to said Secure Digital Flash card, electrically connected to said USB connection, electrically connected to said SDRAM, electrically connected to said Disk On Chip, electrically connected to said keypad and trackball, electrically connected to said wireless link, and electrically connected to said graphics processor.

20. A handheld diagnostic ultrasound system with head mounted display for ultrasound imaging allowing hands free operation comprising:
a handheld, PDA style diagnostic ultrasound device, for processing the diagnostic ultrasound image;
a lcd display, for displaying said diagnostic ultrasound image;
a non-volatile secure digital flash card, for storing multiple diagnostic ultrasound images;
a li-ion battery, for powering the diagnostic ultrasound device;
an usb connection, for cable transfer of the diagnostic ultrasound image to pc or other storage;
a head mounted display, having a wide field of view and audio ear speakers, for viewing the diagnostic ultrasound image in adverse lighting conditions;
an ultrasound probe, for interrogating the tissue in the region of interest, electrically connected to said diagnostic ultrasound device;
a low power, low voltage fpga, for beamforming the diagnostic ultrasound image, electrically connected to said diagnostic ultrasound device;
a rf frame memory, for storing at least one frame of rf data, electrically embedded to said FPGA;
a baseband demodulation, for seperating the incoming rf samples into i (in-phase) and q (quadrature) componets, electrically embedded to said FPGA;
a low power, highly integrated dual-core processor, for control and processing of all diagnsotic ultrasound images, electrically connected to said USB connection, electrically connected to said Secure Digital Flash card, electrically connected to said LCD Display, and electrically connected to said diagnostic ultrasound device;
a low power, low voltage, DDR sdram, for program and data storage of the dual-core processor, electrically connected to said dual-core processor;
a low power, low voltage, non-volatile disk on chip, for storing operating code of the dual core processor, electrically connected to said dual-core processor;
a keypad and trackball, for operator interface, electrically connected to said dual-core processor;
a wireless link, for wireless transfer of the ultrasound image to pc or other storage, electrically connected to said dual-core processor;
a graphics processor, for converting lcd signals into composite ntsc video signals, electrically connected to said dual-core processor, and electrically connected to said head mounted display; and
a lcd controller, for processing the ultrasound image for lcd display.
